(19) United States
(12) Patent Application Publication
(10) Pub. No.: US 2012/0121691 A1
(43) Pub. Date: May 17, 2012

(54) Method for Increasing the Production of a Specific ACYL-Chain Dihydroceramide(s) for Improving the Effectiveness of Cancer Treatments

(75) Inventor: Barry James Maurer, (US)

(73) Assignee: Texas Tech University System

(21) Appl. No.: 13/297,132

(22) Filed: Nov. 15, 2011

Related U.S. Application Data
(60) Provisional application No. 61/413,778, filed on Nov. 15, 2010.

(51) Int. Cl. A61K 31/20; A61P 35/00; A61K 9/127

(52) U.S. Cl. 424/450; 514/558

(57) ABSTRACT

A method to improve the effectiveness of cancer treatments by increasing the production of specific ACYL-chain dihydroceramide(s). Increase of native chain-length dihydroceramides is directly cytotoxic to human acute lymphoblastic leukemia cell line MOLT-4 ALL cells with a cytotoxic potency that is dependent upon the specific fatty acid acyl-chain length and saturation of the dihydroceramides. The combination of sphinganine and GT-11 lead to cell death in the absence of an increase of reactive oxygen species, suggesting that the ability of fenretinide to increase cytotoxic ROS is mechanistically independent of dihydroceramides increase and related cytotoxicity. Most unexpectedly, supplementing the exposure of cancer cells to a dihydroceramide-increasing anti-hyperproliferative agent(s), such as fenretinide, with specifically-chosen fatty acids can increase the cytotoxicity of the anti-hyperproliferative agent to the cancer cells to a beneficial effect.

### CROSS-REFERENCE TO RELATED APPLICATIONS

This Application claims the benefit under Title 35 United States Code §119(e) of U.S. Provisional Application No. 61/413,778, filed Nov. 15, 2010, the full disclosure of which is incorporated herein by reference.

### FIELD OF THE INVENTION

The present invention relates to a method for increasing the production of specific ACYL-chain dihydroceramide(s) for improving the effectiveness of cancer treatments.

### BACKGROUND OF THE INVENTION

Without limiting the scope of the disclosed method, the background is described in connection with a novel method for increasing the production of a specific ACYL-chain dihydroceramide(s) for improving the effectiveness of cancer treatments. Fenretinide (N-(4-hydroxyphenyl)retinamide, 4-HPR) is a cytotoxic retinoid that selectively increases de novo synthesis of dihydroceramides and reactive oxygen species (ROS) in susceptible cancer cell lines, in vitro. High-dose fenretinide has been evaluated clinically against several cancer types, including acute lymphoblastic (ALL) leukemias. Such clinical evaluation demonstrated a correlation between fenretinide-induced increase of dihydroceramides and cytotoxicity, in vitro, however, little was known regarding the direct cytotoxic potency of native, long-chain dihydroceramides.

### SUMMARY OF THE INVENTION

The present invention, therefore, provides a method to improve the effectiveness of cancer treatments by increasing the production of specific ACYL-chain dihydroceramide(s). Increase of native chain-length dihydroceramides is directly cytotoxic to human acute lymphoblastic leukemia cell line MOLT-4 ALL cells with a cytotoxic potency that is dependent upon the specific fatty acid acyl-chain length and saturation of the dihydroceramides in the absence of reactive oxygen species (ROS) increase, with implications for the mechanism of fenretinide cytotoxicity. Native, long-chain dihydroceramides were increased in MOLT-4 ALL cells by exposing them to excess sphinganine in the presence of the ceramide desaturase inhibitor, GT-11. An excess of individual fatty acids was used to bias the production of dihydroceramides of specific acyl chain lengths and saturations. Quantitative data for the dihydroceramide species was correlated with cytotoxicity responses.

Minimally-toxic single agent concentrations of sphinganine and GT-11 induced elevated dihydroceramide levels (up to 18-fold total increase) and caused significant cytotoxicity (−95% cell kill at 24 hours) in MOLT-4 cells. The combination of sphinganine and GT-11 lead to cell death in the absence of an increase of reactive oxygen species, suggesting that the ability of fenretinide to increase cytotoxic ROS is mechanistically independent of dihydroceramides increase and related cytotoxicity. Most unexpectedly, supplementing the exposure of cancer cells to a dihydroceramide-increasing anti-hyperproliferative agent(s), such as fenretinide, with specifically-chosen fatty acids can increase the cytotoxicity of the anti-hyperproliferative agent to the cancer cells to a beneficial effect.

### BRIEF DESCRIPTION OF THE DRAWINGS

For a more complete understanding of the present invention, reference is now made to the detailed description of the invention along with the accompanying figures in which:

FIG. 1 is a schematic illustration of the de novo synthetic pathway of dihydroceramides and ceramides and the structures of these sphingolipids;

FIG. 2 is a bar graph demonstrating that exogenous sphinganine supplementation can be combined with GT-11, a partial inhibitor of dihydroceramide desaturase (DESG-1) to increase dihydroceramides in cancer cell lines, biochemically mimicking the effects of the cytotoxic retinoid, fenretinide (4-HPR) on cancer cell lines;

FIGS. 3A and 3B are 3-axis bar charts demonstrating that exogenous sphinganine combined with GT-11 increased various N-acyl chain dihydroceramides while decreasing the extent of the increase of ceramides observed when sphinganine is used in the absence of the GT-11 inhibitor;

FIGS. 4A-4D are graphs demonstrating that increasing dihydroceramides with sphinganine+GT-11 unexpectedly increases cytotoxicity in cancer cell lines compared to the same concentrations of sphinganine-alone;

FIGS. 5A-5F are 3-axis bar charts demonstrating that cells exposed to sphinganine+GT-11 can be supplemented with exogenous fatty acids to bias the production of specific acyl-chained dihydroceramides;

FIGS. 6A and 6B are graphs demonstrating that, unexpectedly, supplementation of only certain fatty acids results in an increased cytotoxicity of sphinganine+GT-11 in cancer cells;

FIGS. 7A-7F are graphs and 3-axis bar charts demonstrating that, unexpectedly, the increase in cytotoxicity in cancer cells that results from supplementing sphinganine+GT-11 with certain fatty acids correlates with increases in the corresponding N-acyl chain of the dihydroceramide whereas supplementation with other fatty acids resulted in an increase in the corresponding dihydroceramide without an increase in cytotoxicity;

FIGS. 8A-8F are graphs and 3-axis bar charts demonstrating that supplementing fenretinide exposure to cancer cells with certain, but not all, fatty acids can increase fenretinide cytotoxicity;

FIGS. 9A-9D are graphs demonstrating that, unexpectedly, after the manner found in T-cell ALL leukemia cells, supplementation of fenretinide exposure with certain, but not all, fatty acids can increase fenretinide cytotoxicity in a variety of solid tumor cell lines, including colon, breast, and small cell and non-small lung cancers; and

FIGS. 10A and 10B are data plots demonstrating that levels of C22:0 and C24:0 dihydroceramides positively correlated with cytotoxicity in four ALL leukemia cell lines.

### DETAILED DESCRIPTION OF THE INVENTION

### Fatty Acids

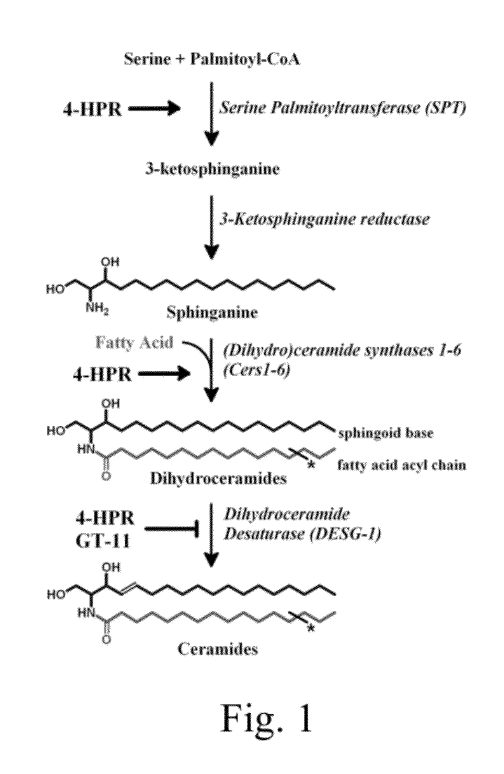

A fatty acid is a carboxylic acid with a long unbranched aliphatic tail (hydrocarbon chain), which is either saturated or unsaturated. Most naturally occurring mammalian fatty acids have a chain of an even number of carbon atoms, from 12 to 28. When they are not attached to other molecules, they are known as “free” fatty acids. Fatty acids that have double bonds are known as unsaturated. Fatty acids without double bonds are known as saturated. Fatty acids differ in length and are often categorized as short, medium, or long; short-chain fatty acids are fatty acids with aliphatic tails of fewer than six carbons (i.e. butyric acid; medium-chain fatty acid are fatty acids with aliphatic tails of 6-12 carbons; long-chain fatty acid are fatty acids with aliphatic tails longer than 12 carbons; very long chain fatty acid are fatty acids with aliphatic tails longer than 22 carbons. Unsaturated fatty acids have one or more (up to six) double bonds between carbon atoms. In most naturally occurring unsaturated fatty acids, each double bond has three n carbon atoms after it and are of cis configuration. The differences in geometry between unsaturated fatty acids, as well as between saturated and unsaturated fatty acids, play an important role in biological processes, and in the construction of biological structures (such as cell membranes). Fatty acids are essential components of sphingolipids. Sphingolipids are ‘wax-like’ molecules built on sphingoid bases and ceramides as shown in FIG. 1.

### Treatment and Administration

The present invention can be administered for the treatment of hyperproliferative disorders such as tumors, cancers and neoplastic disorders, as well as premalignant and non-neoplastic or non-malignant hyperproliferative disorders.

Subjects to be treated by the invention and methods described herein are, in general, mammalian subjects, including both human subjects and animal subjects, such as dogs, cats, horses, etc. for veterinary purposes.

Examples of tumors, cancers, and neoplastic tissue that can be treated by the present invention include but are not limited to malignant disorders such as breast cancers; osteosarcomas; angiosarcomas; fibrosarcomas and other sarcomas; leukemias; lymphomas; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; head and neck cancers; and grain or central and peripheral nervous (CNS) system tumors, malignant or benign, including gliomas and neuroblastomas.

Examples of premalignant and non-neoplastic or non-malignant hyperproliferative disorders include but are not limited to myelodysplastic disorders; cervical carcinoma-in-situ; familial polyposes such as Gardner syndrome; oral leukoplakias; histiocytosis; keloids; hemangiomas; hyperproliferative arterial stenosis, inflammatory arthritis; hyperkeratosis and papulosquamous eruptions including arthritis; viral induced hyperproliferative diseases such as warts and EBV induced dieases, scar formation, and the like. The method of treatment disclosed herein may be employed with any subject known or suspected of carrying or at risk of developing a hyperproliferative disorder as defined herein.

As used herein, “treatment” of a hyperproliferative disorder, such as a cancer, refers to methods of killing, inhibiting or slowing the growth or increase in size of a body or population of hyperproliferative cells, or tumor or cancerous growth, reducing hyperproliferative cell numbers, or preventing spread to other anatomic sites, as well as reducing the size of a hyperproliferative growth or numbers of hyperproliferative cells. As used herein, “treatment” is not necessarily meant to imply cure or complete abolition of the hyperproliferative growths. As used herein, a treatment effective amount is an amount effective to result in the killing, the slowing of the rate of growth of hyperproliferative cells, the decrease in size of a body of hyperproliferative cells, and/or the reduction in the number of hyperproliferative cells, to a greater extent or degree when the specific fatty acid(s) is combined with the anti-hyperproliferative agent(s) than when the anti-hyperproliferative agent(s) are used without the specific fatty acid(s).

The therapeutically effective dose of the specific fatty acid(s) to be administered, the use of which is in the scope of the present inventions, will vary somewhat from subject to subject and will depend upon factors such as the specific condition of the subject in need of treatment, the anti-hyproliferative agent co-administered, and the route of administration. Such dosages can be determined in accordance with routine pharmacological procedures known to those skilled in the art, particularly in light of the disclosure provided herein. For a specific fatty acid(s), a dose to achieve a plasma level of about 1 μM to 10 or 100 μM, or greater, is employed. Daily doses of a specific fatty acid(s) may be 1 g to 10 to 100 g, or greater, is employed. As an example, for an anti-hyproproliferative agent such as fenretinide, a dose to achieve a plasma level of about 5 μM to 10 to 60 μM, or greater, is employed.

The specific fatty acid(s) described herein may be administered by any suitable technique, including orally, intravenously, intraarterially, intramuscularly, subcutaneously, intraperitoneally, intravesicularly, intrathecally, sublingually, or topically, in a continuous or discontinuous manner, either before, concurrently with, or after the anti-hyperproliferative agent.

The specific fatty acids(s) described herein may be administered by any suitable technique including neatly, or compounded in a medicant such as a powder, solution, emulsion, liposome, nanoparticle, organized lipid complex, cream, ointment, gel, or salve. It is understood that the specific fatty acid(s) are incoporated into such preparations in amounts that would not be routine or ordinary practice for the composition of such preparations in the absence of the disclosures of the present invention as described herein.

The specific fatty acid(s) described herein may be co-formulated for delivery with the anti-hyperproliferative agent(s).

The specific fatty acid(s) described herein may be isolated from, or be included in, natural sources, such as vegetable or animal fats or oils or triglycerides, or be synthesized artificially or semi-artificially, which may also be delivered in the form of a triglyceride.

The fatty acid(s) described herein may be used in combination therapies, such as described in B. Maurer et al., U.S. Pat. No. 6,352,844 (Mar. 5, 2002), in B. Maurer et al., U.S. Pat. No. 6,368,831 (Apr. 9, 2002), or with fenretinide formulations such as found in S. Gupta, et al., U.S. Pat. No. 7,169,819 (Jan. 30, 2007), in B. Maurer et al., U.S. Pat. No. 7,785,621 (Aug. 31, 2010), and in B. Maurer et al., U.S. Pat. No. 7,780,978 (Aug. 24, 2010), the disclosure of which is incorporated by reference in its entirety.

The present invention is explained in greater detail in the following non-limiting examples.

### Example 1

### Pathway of De Novo Dihydroceramide Synthesis

FIG. 1 shows a schematic of the de novo sphingolipid pathway. Rate-limiting enzyme serine palmitoyltransferase (SPT) condenses serine and palmitoyl-CoA to 3-ketosphinganine, which is subsequently reduced to sphinganine (the ‘sphingoid base’ or ‘sphingoid backbone’). (Dihydro)ceramide synthases (CerS 1-6) selectively N-acylate sphinganine with a fatty acid acyl-chain that may vary in carbon length and degree of saturation, producing a dihydroceramide. Dihydroceramide desaturase (DEGS-1) desaturates the sphingoid backbone of the dihydroceramide to yield the corresponding ceramide. Fenretinide (4-HPR) is a stimulator of both SPT and CerS. Both 4-HPR and GT-11, a synthetic ceramide derivative, are partial inhibitor of DEGS-1. * denotes variable fatty acyl-chain.

### Example 2

### Cytotoxicity Assay

Materials. Sphinganine [(2S,3R)-2-aminooctadecane-1,3-diol] (Sa) and N-[(1R,2S)-2-hydroxy-1-hydroxymethyl-1-2-(2-tridecyl-1-cyclopropenyl)ethyl]octanamide] (GT-11) were purchased from Avanti Polar Lipids and prepared in ethanol at 10 mM and 1 mM, respectively. Fenretinide [(2E,4E,6E,8E)-N-(4-hydroxyphenyl)-3,7-dimethyl-9-(2,6,6-trimethyl-cyclohexen-1-yl)nona-2,4,6,8-tetraenamide] (4-HPR), was from the National Cancer Institute (NCI) Developmental Therapeutics Program (DTP) of the National Institutes of Health (NIH), and prepared in ethanol (10 mM). Stocks were stored in sealed polypropylene tubes. Fatty acids (Fisher Scientific) were dissolved in solution of methanol/chloroform (1:2, v:v) at 10 mM and stored in PFTE-capped borosilicate vials. Ethanol (200 proof), chloroform (ethanol-stabilized), and other solvents were obtained from Sigma Aldrich or Fisher Scientific. LC/MS/MS solvents were mass spectroscopy grade or higher. Alpha-cyclodextrin (Acros Organics) was dissolved (15 mM) in non-supplemented RPMI-1640 medium (Invitrogen). Sphingolipid standards were obtained from the LIPID MAPS consortium via Avanti Polar Lipids. Radiolabeled fatty acids were purchased from American Radiolabeled Chemicals.

Cell culture. The pre-T acute lymphoblastic leukemia cell lines MOLT-4 and CCRF-CEM, small cell lung cancer cell line NCI-H146, non-small cell lung cancer cell line NCI-H1792, colon cancer cell lines LoVo and HT-29, and breast cancer cell line MCF-7, were purchased from American Type Culture Collection, Manassas, Va., and grown at 20%/5% and 5%/5%, respectively. COG-LL-317 and COG-LL-332 pre-T acute lymphoblastic leukemia cell lines were obtained from the TTUHSC Cancer Center Cell Repository and grown at 5%/5%. Cell line identities were verified by short tandem repeat analysis and mycoplasma testing was performed. Cell lines were maintained in RPMI-1640 medium supplemented with 10% fetal bovine serum (FBS, Invitrogen) in humidified, 37° C. incubators. For all experiments, ALL cell lines were seeded at 2. cells/mL in RPMI-1640 medium supplemented with 15% fetal bovine serum. Solid tumor cell lines were plated at 5×cells/mL in RPMI-1640 medium supplemented with 10% fetal bovine serum.

Fatty acid solubilization. Fatty acids were solubilized using a modified protocol from Singh and Kishimoto (23). Briefly, fatty acid was added to a sterile, glass Erlenmeyer flask and dried under nitrogen. A solution of α-cyclodextrin (15 mM/RPMI-1640) was then added at 27.3 mL/μmol FA. The well-sealed flask was then sonicated thrice for 5 minutes each using a Branson 2510 Bath Sonicator (30° C.). The fatty acid solution was then sterilized by filtration (Millipore 0.22 μm PVDF filter) and diluted by one-fourth with RPMI-1640 medium. The resulting solubilized fatty acid concentration was determined with —C22:0- and —C24:0-fatty acids to be 15 μM. The final concentration of fatty acid in cell culture was 5 μM.

Cytotoxicity Assay. Cytoxicity is determined using the DIMSCAN assay system (R. Proffitt et al., Cytometry 24, 204-213 (1996); T. Frgala et al., Proc. AACR, 36, 303 (1995). The system employs digital imaging microscopy to quantify viable cells, which selectively accumulate fluorescein diacetate to become brightly fluorescent. The system is capable of measuring cytotoxicity over a 4-5 log dynamic range by quenching the residual fluorescence of dead and dying cells with eosin Y and quantifying the total fluorescence of viable cells using digital thresholding. Measured fluorescence is directly proportionate to the number of viable cells. A comparison of the total fluorescence of a drug-treated cell population to the fluorescence of a similar number of untreated cells yields a survival fraction.

In brief, 5,000 to 25,000 cells/well depending on the cell line (5,000 for solid tumor, 25,000 for ALL cell lines) were replicate plated into 60 wells of a 96-well tissue culture plate in 0.1 cc media and allowed to attach or recover overnight. Drug(s) are then added in 0.05 cc media to the final concentrations indicated. There are 12 wells treated per drug concentration. Twelve wells receive drug-vector only to the appropriate final concentration and serve as controls for the plate. Cells are incubated for 48-96 hours at 37° C. in 5%. Fluorescein diacetate is then added to each well in 0.05 cc media to a final concentration of 8 microgram/cc. Cells are incubated for a further 15 minutes at 37° C. and 0.03 cc of 0.5% eosin Y is added to each well. Total fluorescence of viable cells is then measured by digital imaging microscopy and the signal normalized to control cells.

### Example 3

### Sphingolipid Assay

LC/MS/MS analysis of intracellular sphingolipids. Sphingolipids were separated using an Agilent 1200 HPLC (LC) and determined by ESI/MS/MS performed on a AB SCIEX 4000 QTRAP Hybrid Triple Quadrupole/Linear Ion Trap mass spectrometer (MS), operating in a multiple reaction monitoring positive ionization mode as described previously with moderate modifications (25). Briefly, 50 μL of a solution (1 μM) of internal sphingolipid standards (including -sphingosine, -sphinganine, -sphingosine-1-phosphate, and -ceramide) was added to each cell pellet sample. Lipids of each sample were extracted twice with 2 mL of the ethyl acetate/isopropyl alcohol/water (60:28:12; v:v) solvent system. Supernatants were transferred to glass tubes (Kimble Chase) and evaporated under air (10 PSI) at 40° C. After reconstitution in methanol (4 mL), 1 ml of each sample was separated for the determination of lipid phosphate. Remaining sample (3 mL) was dried and used for sphingolipid quantification. For ESI/MS/MS, the dried lipid sample was dissolved in mobile phase A. Samples were injected (10 μL) and separated on a Spectra C8SR, 150×3.0 mm, 3-μm particle size column using gradient-elution (mobile phase A/B, prepared as previously described).

Data acquisition, peak integration and analyte quantitation were performed using ABI/SCIEX Analyst 1.4.2 Software. Sphingolipid data were normalized to lipid phosphate as previously described (26). Briefly, samples were dried under air (10 PSI) and lipids were extracted using the method of Bligh and Dyer (27). Of importance for phosphate assay, only Kimble Chase disposable borosilicate tubes were used for minimal sample contamination. Organic phase was transferred to glass tubes and a known volume was separated to a new tube and dried at 80° C. Phosphate standards and dried samples were then heated with ashing buffer (water:10 N:70% [40:9:1]) at 160° C. overnight. Samples were then incubated with ammonium molybdate and ascorbic acid as previously described, and absorbance (820 nM) was read using a SpectraMax.

Statistical analyses. Descriptive statistics and significance testing were performed using Microsoft Excel. The Excel function “t.test” was used for Student's T-testing (two-tailed, type-2). Propagated standard deviations were calculated using standard methods. Correlation analyses were performed using Sigmaplot 11 software.

### Example 4

### Sphinganine+GT-11 Increased Dihydroceramides

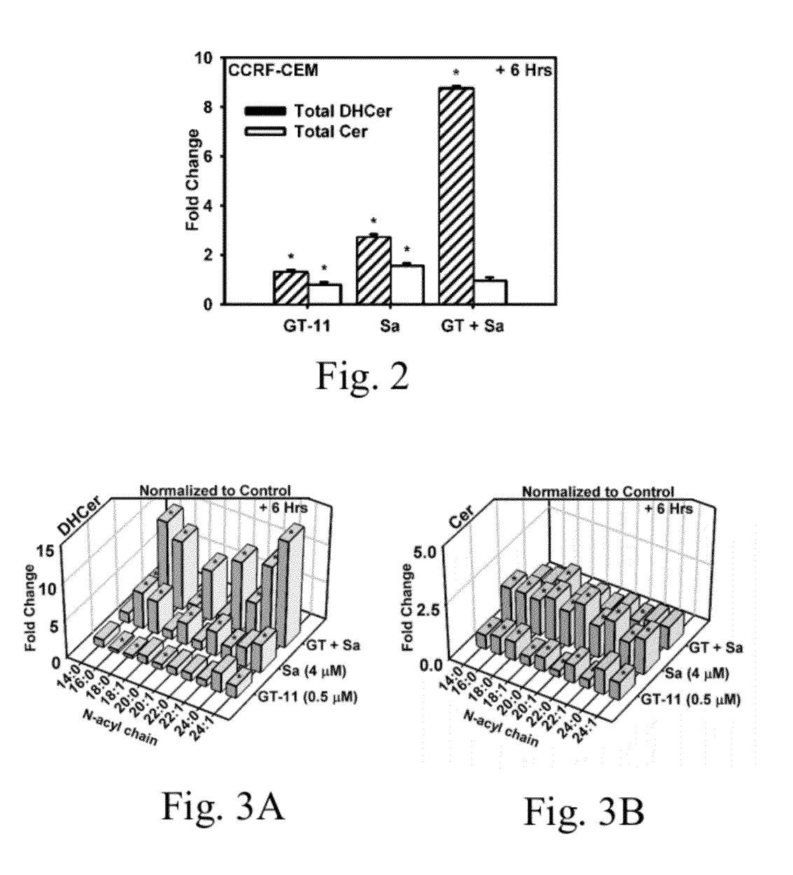

Sphinganine (Sa) is the immediate sphingoid base/backbone precursor to dihydroceramides (DHCer) which is acylated to various carbon chain length fatty acids to form dihydroceramides as shown in FIG. 1. GT-11 is a partial inhibitor of dihydroceramide desaturase (DEGS-1) which desaturates the sphinganine backbone of the dihydroceramide to yield the corresponding ceramide. As shown in FIG. 2, sphinganine co-treatment with GT-11 of CEM T-cell ALL cells increased total dihydroceramides at +6 hrs by increasing dihydroceramides synthesis and decreasing the conversion of dihydroceramides to ceramides. Total dihydroceramides and ceramides were normalized to control and plotted as fold change (bar, y-axis) in FIG. 2. Also in FIG. 2: error bar, propagated SD, * indicates significance, with P<0.001.

### Example 5

### Sphinganine+GT-11 Increased Dihydroceramides

As shown in FIG. 1, sphinganine (Sa) is the immediate sphingoid base/backbone precursor to dihydroceramides (DHCer) which is acylated to various carbon chain length fatty acids to form dihydroceramides. GT-11 is a partial inhibitor of dihydroceramide desaturase (DEGS-1) which desaturates the sphinganine backbone of the dihydroceramide to yield the corresponding ceramide. Treatment with sphinganine (Sa) plus GT-11 differentially increased dihydroceramides (DHCer) as shown in FIG. 3A, while sphinganine alone differentially increased ceramides (Cer) in CEM T-cell ALL cells at +6 hrs, see FIG. 3B. Sphingolipids were normalized to control and plotted as fold change (bar, z-axis). N-acyl chain (x-axis) corresponds to carbon length and degree of saturation in acyl portion of sphingolipid (* indicates significance, with P<0.05).

CCRF-CEM cells were treated with drug/fatty acid vehicles (control), GT-11 (0.5 μM) alone, sphinganine (4 μM) alone or sphinganine (4 μM) plus GT-11 (0.5 μM) for six hours and subsequently prepared for sphingolipid analysis. Increased native-acyl chain dihydroceramides resulted from de novo sphingolipid pathway modulation. Fenretinide has reported cytotoxicity in several pre-T acute lymphoblastic leukemia cell lines in vitro, in association with increased production of de novo dihydroceramide (3). However, artificial, cell-penetrant, short-acyl chain dihydroceramides have been reported to be minimally cytotoxic in HL-60 acute myelogenous leukemia cells (19). Therefore, to investigate the cytotoxic potential of the native long- and very long-acyl chain dihydroceramides increased by fenretinide, we mimicked the fenretinide-induced increase of native dihydroceramides though de novo synthesis.

Exogenous addition of sphinganine (the product of SPT) and GT-11, a DEGS-1 inhibitor, were used to specifically increase dihydroceramide synthesis and accumulation. Treatment of CCRF-CEM cells with sphinganine (4 μM) and GT-11 (0.5 μM) for six hours resulted in an 8.8-fold increase (P<0.001) of total dihydroceramide (see FIG. 2), including significant increases (P<0.05) of each dihydroceramide analyte except C20:1-DHCer as shown in FIG. 3A. Sphinganine alone resulted in a 1.5 fold increase (P<0.001) of total ceramide (see FIG. 2), including significant increases (P<0.05) of all ceramide analytes (see FIG. 3B).

### Example 6

### Dihydroceramide Levels Associate with Cytotoxicity

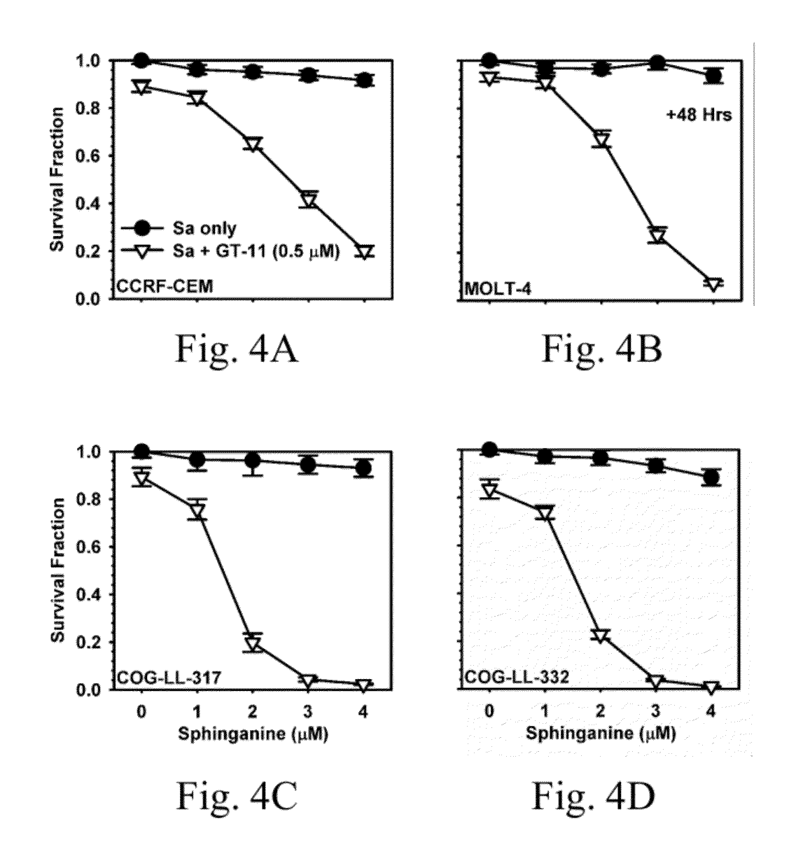

Increased dihydroceramide levels (FIG. 3) were associated with increased cytotoxicity in acute lymphoblastic leukemia cell lines (FIG. 4). The pre-T acute lymphoblastic leukemia cell lines CCRF-CEM, MOLT-4, COG-LL-317 and COG-LL-332 were treated for 48 hours with sphinganine (0-4 μM) with and without GT-11 (0.5 μM)(FIG. 4). Cytotoxicity was then measured using DIMSCAN cytotoxicity analysis. Endpoint cytotoxicity data was normalized to control cells that received drug/fatty acid vehicle and plotted as survival fraction (y-axis). Datum point, mean; error bar, SEM.

Cytotoxicity was associated with elevated levels of native dihydroceramides. To evaluate the cytotoxicity associated with elevated dihydroceramides, cells were treated with individually non-cytotoxic concentrations of sphinganine and GT-11. The combination of sphinganine (4 μM) plus GT-11 (0.5 μM) resulted in a 76 to 96 percent increase in cytotoxicity over sphinganine alone, across the four cell lines (FIG. 4). Cytotoxicity induced by sphinganine plus GT-11 increased in a sphinganine dose-dependent manner in each cell line.

### Example 7

### Fatty Acid Co-Treatment Resulted in Targeted Dihydroceramide Production Bias

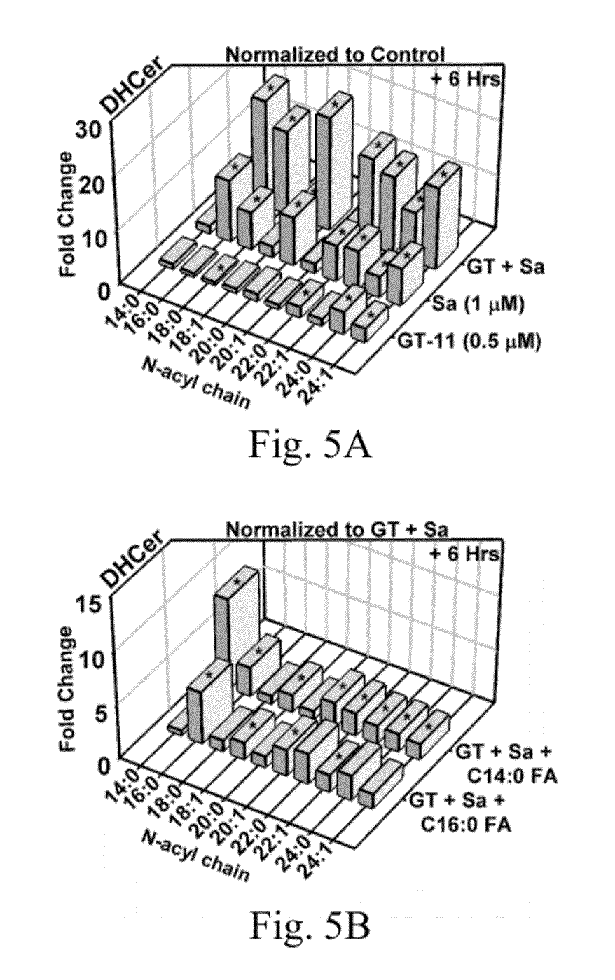

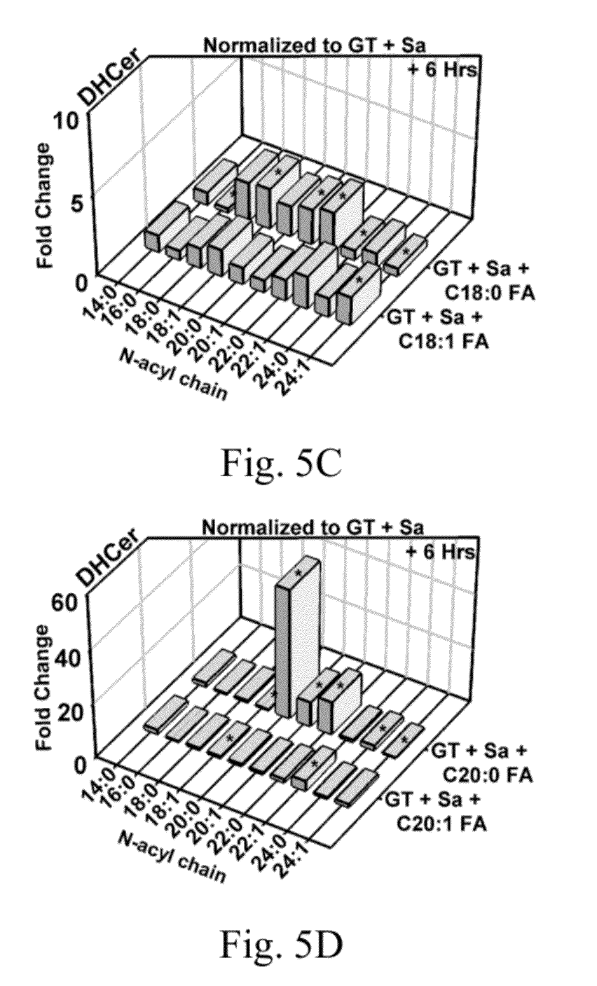

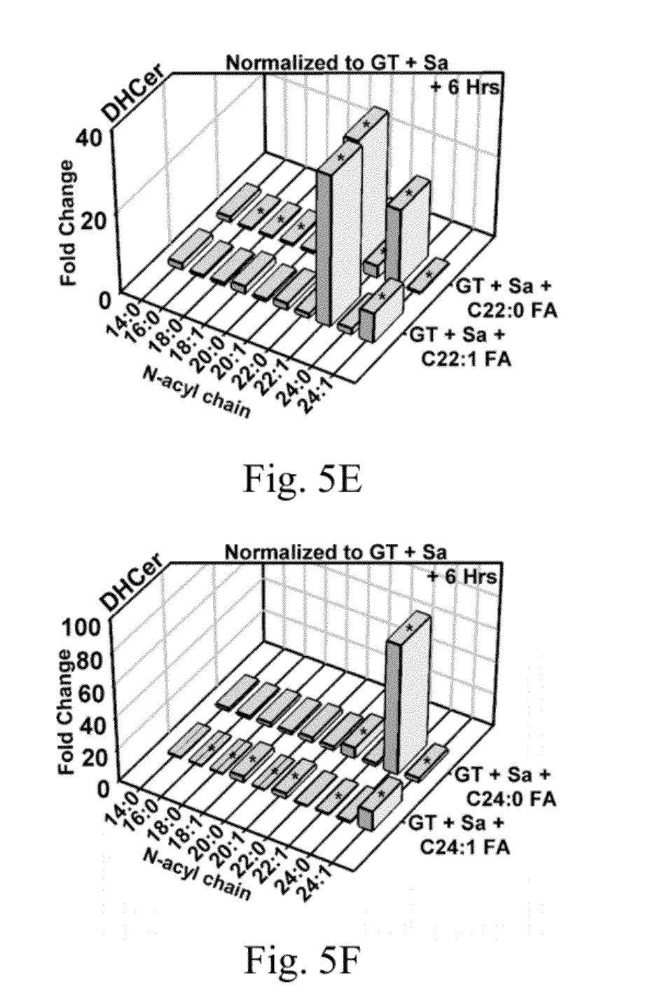

Specific fatty acid co-treatment with sphinganine (Sa) and GT-11 resulted in a biased increase of targeted dihydroceramide (DHCer), i.e., a preferential incorporation of the supplemented fatty acid into the acyl chain of the dihydroceramides increased (FIG. 5A-F).

FIG. 5A: treatment of CCRF-CEM ALL cells with sphinganine (Sa) and GT-11 resulted in differentially increased dihydroceramide levels depending on the fatty acid supplemented. CCRF-CEM cells were treated with GT-11 (0.5 μM) alone, sphinganine (1 μM) alone or sphinganine (1 μM) plus GT-11 (0.5 μM) for six hours and prepared for quantitative analysis. DHCers were normalized to control and plotted as fold change (bar, z-axis). * indicates significance, with P<0.05. FIGS. 5B-5F: solubilized fatty acids biased sphinganine/GT-11 driven dihydroceramides for respective N-acyl chains. CCRF-CEM cells were treated with sphinganine (1 μM) plus GT-11 (0.5 μM) with and without specific solubilized fatty acids (carbon chain length (CX) and saturation (:X), Ex: C20:1=fatty acid with 20 carbons and one desaturation) for six hours and prepared for quantitative sphingolipid analysis. Dihydroceramides were normalized to control cells that received sphinganine plus GT-11 with no fatty acid and plotted as fold change (bar, z-axis). * indicates significance, with P<0.05.

De novo sphingolipid production was biased by addition of specific fatty acids. Although a positive association of dihydroceramide accumulation and cytotoxicity was observed, the cytotoxic properties of constituent dihydroceramides are likely defined by the acyl-chain carbon length and degree of saturation. To bias de novo synthesis for specific sphingolipids, cells were treated with solubilized fatty acid in addition to sphinganine with and without GT-11 to increase specific dihydroceramide and ceramide levels, respectively. CCRF-CEM cells were treated for six hours with sphinganine and GT-11, in addition to specific solubilized fatty acids (C14:0, C16:0, C18:0, C18:1, C20:0, C20:1, C22:0, C22:1, C24:0 or C24:1). C14:0-, C16:0-, C20:0-, C22:0, C22:1-, C24:0-, and C24:1-fatty acids in addition to sphinganine plus GT-11 led to significant increases (P<0.05) in corresponding dihydroceramides over sphinganine plus GT-11 alone (FIG. 5). Elongation by two carbon units of some fatty acids led to increased levels of other dihydroceramides. A representative observation was that C22:0-fatty acid in addition to sphinganine plus GT-11 treatment in CCRF-CEM resulted in a 32-fold increase in C22:0-DHCer (P<0.001) and a 19-fold increase in C24:0-DHCer (P<0.001) (FIG. 5E) over sphinganine plus GT-11 alone. Total ceramide levels increased with sphinganine treatment in the absence of desaturase inhibitor GT-11 (not shown). C22:0-fatty acid in addition to sphinganine treatment in CCRF-CEM resulted in a 13-fold increase in C22:0-Cer (P<0.001) and a 4.6-fold increase in C24:0-Cer (P<0.001) over sphinganine alone (not shown).

### Example 8

### Biased Increase of Specific Dihydroceramides Increased Cytotoxicity

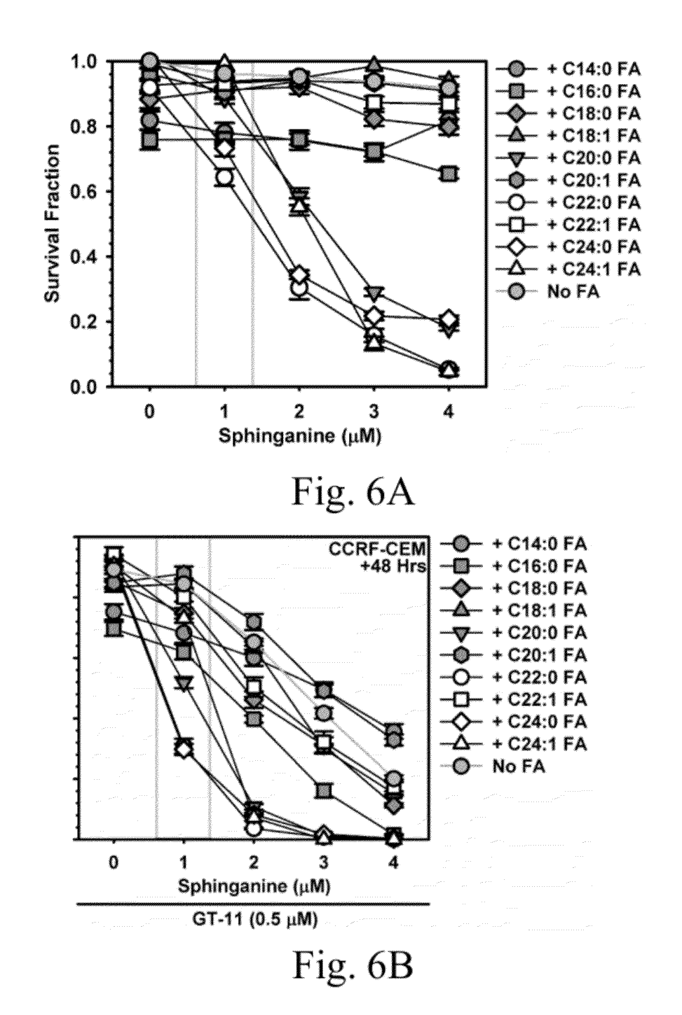

Bias of sphingolipid production with specific fatty acids resulted in differential cytotoxicity (FIGS. 6A and 6B). FIG. 6A: Cytotoxicity of CCRF-CEM cells treated with sphinganine (Sa) and various fatty acids. FIG. 6B: Cytotoxicity of CCRF-CEM cells treated with sphinganine (Sa) plus GT-11 and various fatty acids. CCRF-CEM cells were treated for 48 hours with Sa (0-4 μM)+/−GT-11 (0.5 μM) with or without indicated solubilized fatty acids. Endpoint cytotoxicity was measured using DIMSCAN, and data was normalized to control and plotted as survival fraction (y-axis). Gray and white symbols represent long and very long chain fatty acids, respectively. Gray lines encompass data points used for sphingolipid correlation analysis with cytotoxicity. Datum point, mean; error bar, SEM.

Sphingolipid bias with specific fatty acids resulted in differential cytotoxicity. To evaluate the cytotoxic response of CCRF-CEM to biased sphingolipid production, cells were treated for 48 hours with sphinganine+/−GT-11, in addition to specific solubilized fatty acids (C14:0, C16:0, C18:0, C18:1, C20:0, C20:1, C22:0, C22:1, C24:0 or C24:1). The cytotoxic response of CCRF-CEM to sphinganine or sphinganine plus GT-11 significantly varied depending upon the fatty acid added (FIGS. 6A and 6B). Increases in cytotoxicity occurred in a sphinganine dose-dependent manner. Although large differences in cytotoxicity occurred, fatty acid inter-conversion necessitated the use of correlation analyses to identify cytotoxic species (see Example 11, Tables 1-5).

### Example 9

### Only Certain Acyl-Chain Length Dihydroceramides Increased Cytotoxicity

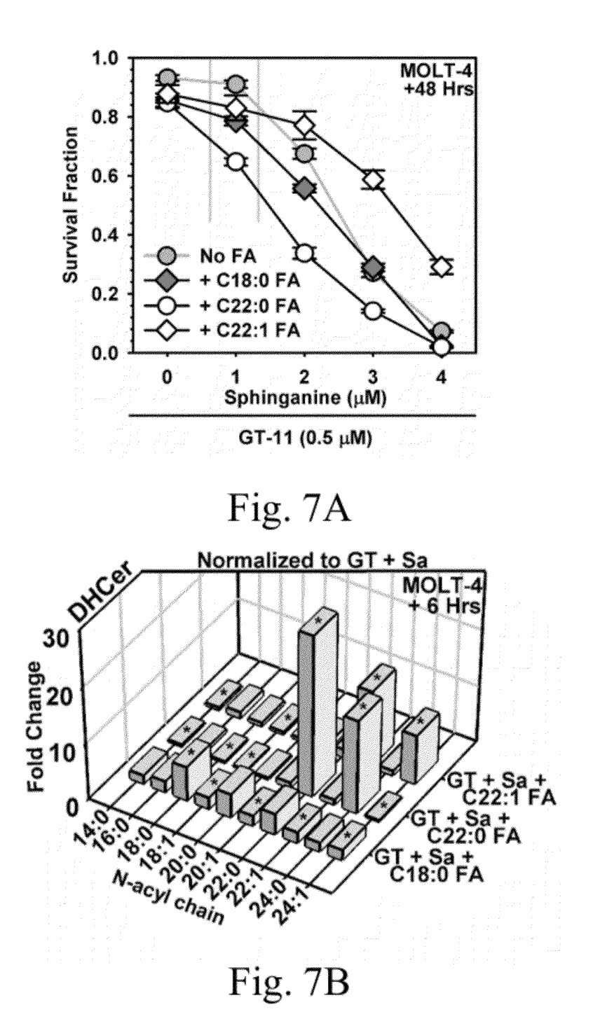

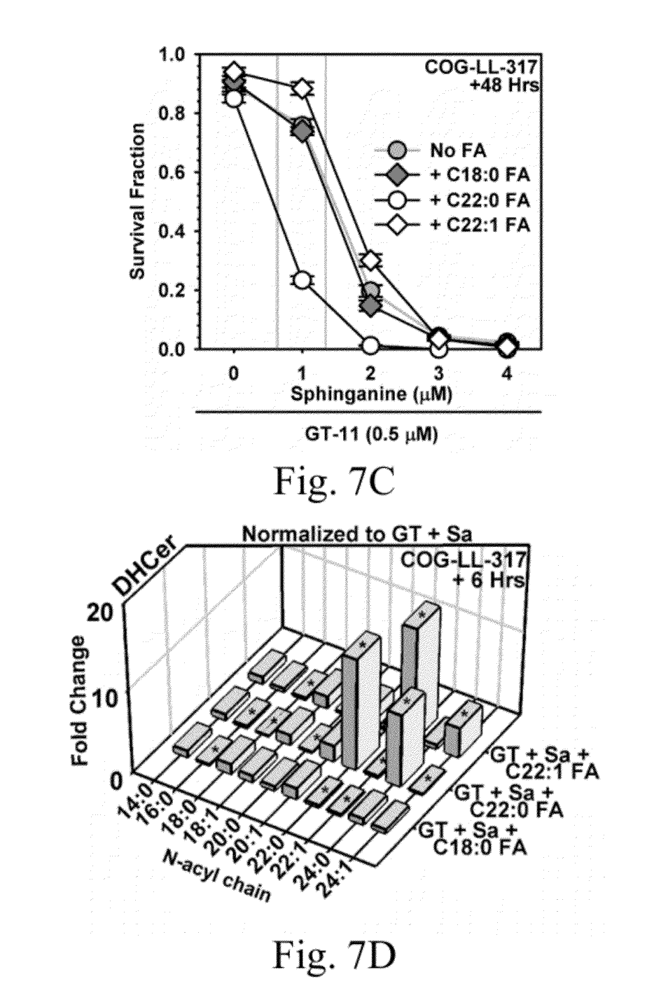

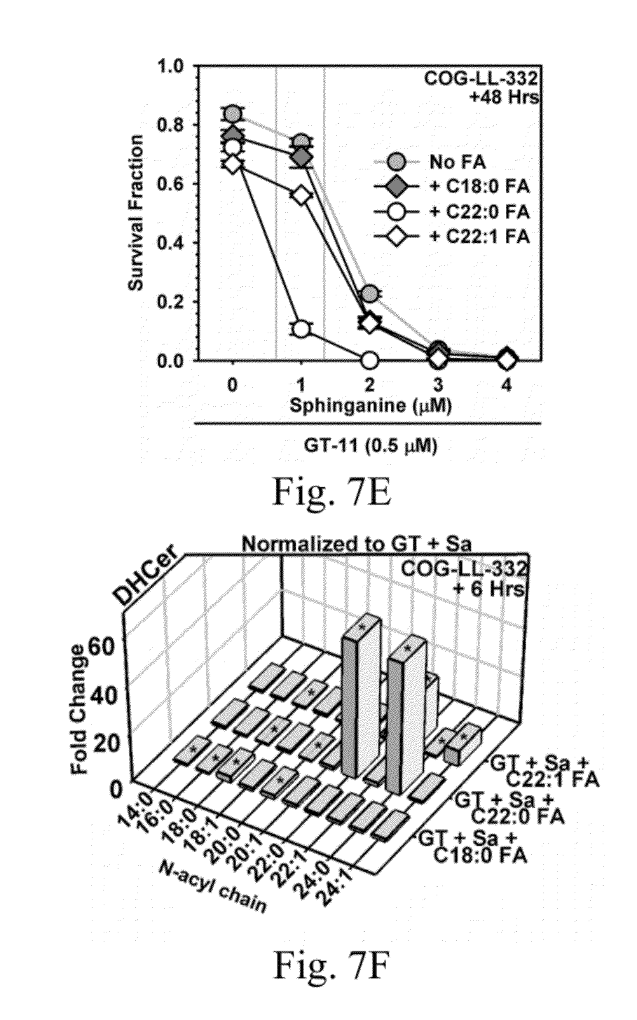

The addition of C22:0-fatty acid, but not C18:0 or C22:1 fatty acids, increased cytotoxicity (FIGS. 7A-7F). C22:0-fatty acid in addition to sphinganine (Sa) plus GT-11 increased cytotoxic C22:0- and C24:0-dihydroceramides in pre-T acute lymphoblastic leukemia cell lines. Cytotoxicity of sphinganine (Sa) (1 μM) with GT-11 was increased with co-treatment of C22:0-fatty acid in MOLT-4 (FIG. 7A), COG-LL-317 (FIG. 7C) and COG-LL-332 (FIG. 7E). Cells were treated for 48 hours with Sa (0-4 μM)+/−GT-11 (0.5 μM) with or without C18:0-, C22:0- or C22:1-fatty acids. Cytotoxicity was measured using DIMSCAN, and data was normalized to control and plotted as survival fraction (y-axis). Gray and white symbols represent long and very long chain fatty acids, respectively. Gray lines encompass data used for sphingolipid correlation analysis with cytotoxicity. Datum point, mean; error bar, SEM. FIG. 7B, FIG. 7D, FIG. 7F: GT-11 and sphinganine (Sa)-driven dihydroceramide synthesis in the presence of C22:0-fatty acid increased C22:0- and C24:0-dihydroceramide levels. MOLT-4 (FIG. 7B), COG-LL-317 (FIG. 7D) and COG-LL-332 (FIG. 7F) were treated with sphinganine (1 μM) plus GT-11 (0.5 μM) with and without C18:0-, C22:0- or C22:1-fatty acids for six hours and subsequently prepared for sphingolipid analysis. Dihydroceramides were normalized to cells that received sphinganine (Sa)(1 μM) plus GT-11 (0.5 μM) with no fatty acid and plotted as fold change (bar, z-axis). * indicates significance, with P<0.05.

C22:0- and C24:0-dihydroceramide levels positively correlated with cytotoxicity in MOLT-4, COG-LL-317 and COG-LL-332. Experimental observations from the CCRF-CEM cell line were used in the selection of a subset of fatty acids for testing in additional pre-T acute lymphoblastic leukemia cell lines. C22:0-fatty acid was utilized to bias the sphingolipid production for C22:0- and C24:0-dihydroceramides. C22:1-fatty acid was utilized as a negative control for C22:0-fatty acid, because while C22:1-dihydroceramide levels reached a similar magnitude as C22:0-dihydroceramide, C22:1-fatty acid did not impart any additional cytotoxicity. MOLT-4, COG-LL-317 and COG-LL-332 cells were treated for six hours with sphinganine+/−GT-11, in addition to solubilized C18:0-, C22:0- and C22:1-fatty acids.

In the three cell lines tested, sphinganine+GT-11 with both C22:0- and C22:1-fatty acids led to significant increases (P<0.05) in the corresponding dihydroceramides over sphinganine+GT-11 alone (FIGS. 7B, 7D and 7F). Conversion of C22:0- to C24:0-fatty acid as well as conversion of C22:1- to C24:1- was observed, as indicated by elevation of C24:0- and C24:1-dihydroceramides, respectively. C22:0-fatty acid in addition to sphinganine+GT-11 treatment increased C22:0-dihydroceramide 29-fold (P<0.01), 13-fold (P<0.003), and 57-fold (P<0.001) in MOLT-4, COG-LL-317, and COG-LL-332, respectively. Significantly increased cytotoxicity was consistently observed in cells treated with C22:0-fatty acid combined with sphinganine (1 μM) and GT-11. MOLT-4, COG-LL-317 and COG-LL-332 also demonstrated a sphinganine dose-dependent increase in cytotoxicity with treatment of C22:0-fatty acid+sphinganine alone. Non-parametric correlation analysis of quantitative and cytotoxicity data independently for each cell line revealed a significant, very strong positive correlation between cytotoxicity and absolute levels of both C22:0- and C24:0-dihydroceramides (see Example 11, Tables 1-5). No consistent correlations were observed between cytotoxicity and total dihydroceramide, total ceramide, or sphingoid base (sphinganine, sphinganine-1-P, sphingosine, sphingosine-1-P) levels (see Example 11, Tables 1-5). NOTE: fatty acid elongase enzymes in cells metabolize a certain quantity of the exogenously supplemented C22:0 fatty acid into C24:0 fatty acids within the cell.

### Example 10

### Specific Fatty Acids Increased Fenretinide (4-HPR) Cytotoxicity in Association with an Increase in Levels of the Corresponding Dihydroceramide

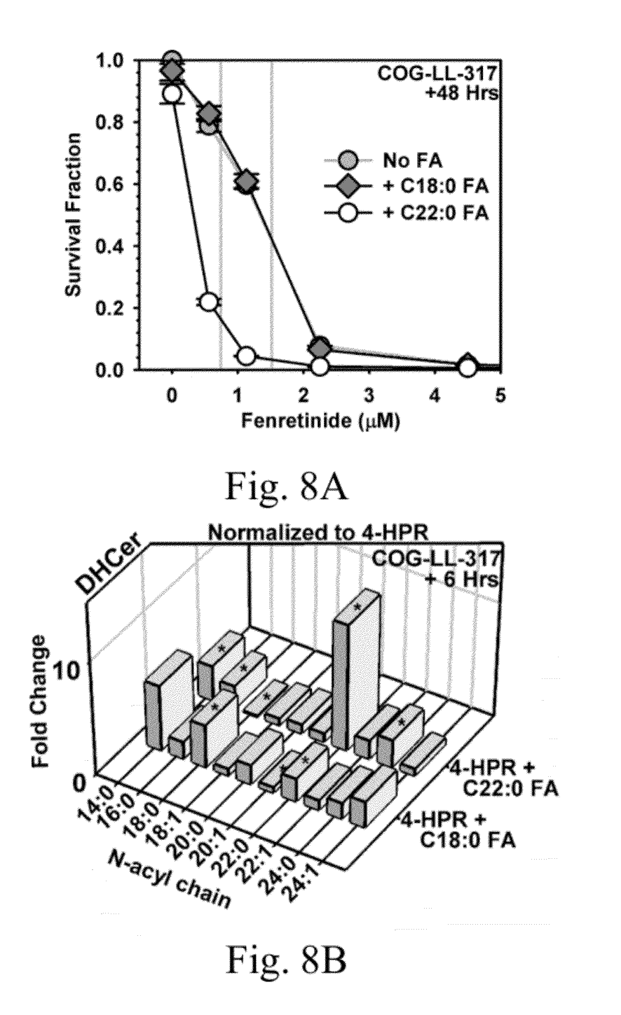

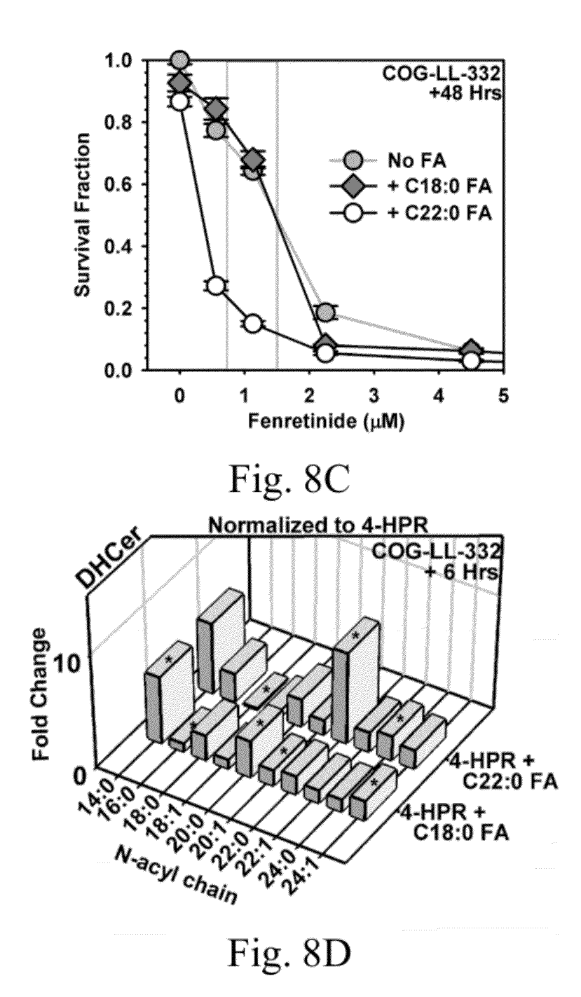

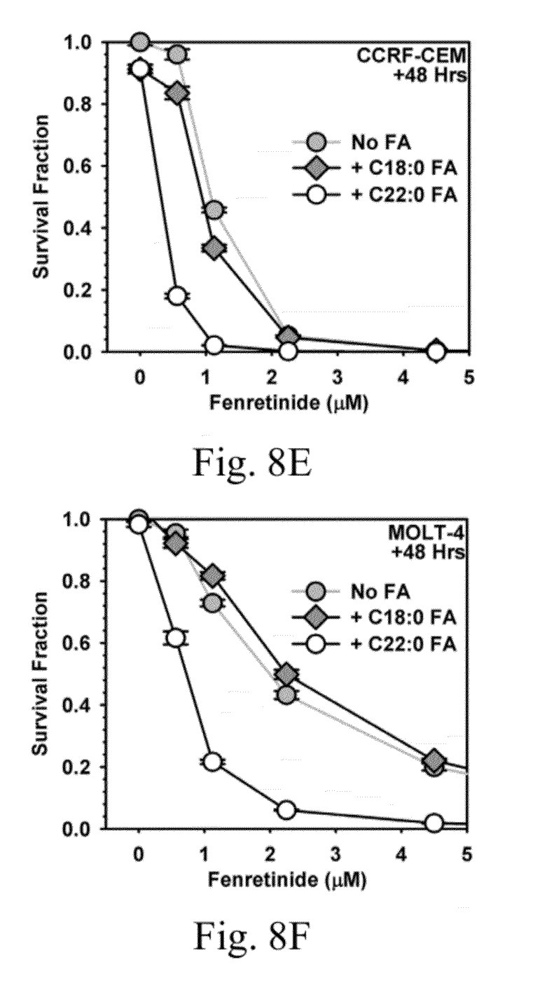

C22:0-fatty acid, but not C18:0 fatty acid, enhanced fenretinide cytotoxicity through increased C22:0-dihydroceramide levels (FIGS. 8A-8F). FIG. 8A: COG-LL-317, FIG. 8B: COG-LL-332, FIG. 8E: CCRF-CEM, and FIG. 8F: MOLT-4 ALL cell lines were treated for 48 hours with 4-HPR (0-9 μM) with or without C18:0- or C22:0-fatty acids. Cytotoxicity was measured using DIMSCAN, and data was normalized to control and plotted as survival fraction (y-axis). Gray lines encompass data used for correlation analysis. Datum point, mean; error bar, SEM. FIG. 8B: COG-LL-317, and FIG. 8D: COG-LL-332 ALL cell lines were treated with 4-HPR (1.1 μM) with or without C18:0- or C22:0-fatty acids for six hours and subsequently prepared for sphingolipid analysis. Dihydroceramides were normalized to cells that received 4-HPR with no fatty acid and plotted as fold change (bar, z-axis). * indicates significance, with P<0.05. Previous results demonstrated both the cancer-specific modulation of de novo sphingolipid synthesis by 4-HPR, and the associated increase in dihydroceramide levels.

Across a panel of pre-T acute lymphoblastic leukemia cell lines, C22:0- and C24:0-dihydroceramides were positively correlated with cytotoxicity. Utilizing solubilized C22:0-fatty acid, 4-HPR-induced dihydroceramide synthesis was biased specifically for C22:0- and C24:0-dihydroceramides. C18:0- and C22:0-fatty acids were administered with fenretinide to COG-LL-317 and COG-LL-332 cell lines. C22:0-fatty acid with 4-HPR led to an 11-fold (P<0.001) and 8-fold (P<0.001) increase of C22:0-dihydroceramide in COG-LL-317 (FIG. 8B) and COG-LL-332 (FIG. 8D), respectively, compared to 4-HPR alone. Next, the cytotoxicity of 4-HPR−/+C18:0- or C22:0-fatty acids was determined. The combination of C22:0-fatty acid with low doses of 4-HPR significantly increased cytotoxicity in each the cell lines tested (FIGS. 8A-8F). NOTE: fatty acid elongase enzymes in cells metabolize a certain quantity of the exogenously supplemented C22:0 fatty acid into C24:0 fatty acids within the cell.

TABLE 1
SUMMARY TABLE - Correlation Analysis

| | |
|---|---|
| C22-DHCer | C24-DHCer |

| Cell Line | ρ | P-value | ρ | P-value |
|---|---|---|---|---|
| CCRF-CEM | 0.75 | <0.001 | 0.84 | <0.001 |
| MOLT-4 | 0.91 | <0.001 | 0.88 | <0.001 |
| COG-LL-317 | 0.79 | <0.02 | 0.91 | <0.001 |
| COG-LL-332 | 0.81 | <0.02 | 0.83 | <0.02 |

TABLE 2A
CRRF-CEM

| | DHCer N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | 0.12 | 0.26 | 0.22 | −0.25 | 0.20 | 0.02 | 0.67 | −0.13 | 0.58 | −0.17 |
| Coef. (r) | | | | | | | | | | |
| p-value | 0.59 | 0.24 | 0.34 | 0.27 | 0.38 | 0.94 | 6.8 × 10⁻⁴ | 0.56 | 4.7 × 10⁻³ | 0.45 |
| Spearman | 0.20 | 0.37 | 0.15 | −0.18 | 0.32 | 0.13 | 0.75 | 0.22 | 0.84 | −0.07 |
| Coef. (ρ) | | | | | | | | | | |
| p-value | 0.37 | 0.08 | 0.50 | 0.41 | 0.15 | 0.57 | 2.0 × 10⁻⁷ | 0.33 | 2.0 × 10⁻⁷ | 0.75 |

TABLE 2B
CRRF-CEM

| | Cer N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | 0.03 | 0.25 | 0.14 | 0.11 | 0.01 | −0.06 | 0.42 | −0.23 | 0.40 | −0.27 |
| Coef. (r) | | | | | | | | | | |
| p | 0.89 | 0.26 | 0.53 | 0.64 | 0.98 | 0.79 | 0.05 | 0.29 | 0.06 | 0.23 |
| Spearman | 0.01 | 0.03 | 0.07 | 0.10 | 0.07 | −0.08 | 0.48 | −0.24 | 0.41 | −0.32 |
| Coef. (ρ) | | | | | | | | | | |
| p | 0.97 | 0.89 | 0.76 | 0.64 | 0.76 | 0.73 | 0.02 | 0.28 | 0.06 | 0.14 |

TABLE 2C
CRRF-CEM

| | Total | Total | | | | |
|---|---|---|---|---|---|---|
| | DHCer | Cer | Sphinganine | Sphingosine | Sphingaine-1-P | Sphingosine-1-P |
| Pearson | 0.26 | 0.07 | −0.21 | 0.02 | 0.44 | −0.07 |
| Coef. (r) | | | | | | |
| p | 0.24 | 0.75 | 0.35 | 0.94 | 0.04 | 0.77 |
| Spearman | 0.42 | −2.8 × 10⁻³ | −0.30 | −0.19 | 0.38 | 0.27 |
| Coef. (ρ) | | | | | | |
| p | 0.051 | 0.99 | 0.17 | 0.38 | 0.08 | 0.23 |

TABLE 3A
COG-LL-317

| | DHCer N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | 0.16 | 0.03 | −0.22 | 0.48 | −0.04 | −0.39 | 0.86 | −0.28 | 0.89 | −0.29 |
| Coef. (r) | | | | | | | | | | |
| p-value | 0.71 | 0.95 | 0.60 | 0.23 | 0.92 | 0.34 | 6.6 × 10⁻³ | 0.50 | 3.3 × 10⁻³ | 0.49 |
| Spearman | 0.10 | 0 | −0.38 | 0.48 | 0.14 | −0.33 | 0.79 | −0.26 | 0.91 | −0.21 |
| Coef. (ρ) | | | | | | | | | | |
| p-value | 0.79 | 0.98 | 0.32 | 0.21 | 0.71 | 0.39 | 1.5 × 10⁻² | 0.50 | 2.0 × 10⁻⁷ | 0.58 |

TABLE 3B
COG-LL-317

| | Ceramide N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | −0.54 | −0.48 | −0.45 | −0.50 | −0.63 | −0.46 | 0.59 | −0.28 | 0.48 | −0.42 |
| Coef. (r) | | | | | | | | | | |
| p | 0.17 | 0.23 | 0.27 | 0.21 | 0.09 | 0.26 | 0.12 | 0.50 | 0.23 | 0.30 |
| Spearman | −0.43 | −0.50 | −0.60 | −0.59 | −0.81 | −0.60 | 0.33 | −0.60 | 0.25 | −0.74 |
| Coef. (ρ) | | | | | | | | | | |
| p | 0.26 | 0.18 | 0.10 | 0.10 | 0.01 | 0.10 | 0.39 | 0.10 | 0.50 | 0.03 |

TABLE 3C
COG-LL-317

| | Total | Total |
|---|---|---|
| | DHCer | Ceramide |
| Pearson | 0.25 | 0.03 |
| Coef. (r) | | |
| p | 0.55 | 0.94 |

TABLE 4A
MOLT-4

| | DHCer N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | 0.13 | 0.26 | 0.12 | 0.04 | 0.12 | −0.37 | 0.79 | −2.5 × 10⁻³ | 0.79 | −0.04 |
| Coef. (r) | | | | | | | | | | |
| p-value | 0.76 | 0.53 | 0.77 | 0.93 | 0.78 | 0.37 | 1.9 × 10⁻² | 1.00 | 2.0 × 10⁻² | 0.93 |
| Spearman | 0.35 | 0.57 | 0.14 | 0.10 | 0.14 | −0.52 | 0.91 | 0.24 | 0.88 | 0.12 |
| Coef. (ρ) | | | | | | | | | | |
| p-value | 0.35 | 0.12 | 0.71 | 0.79 | 0.71 | 0.16 | 2.0 × 10⁻⁷ | 0.54 | 2.0 × 10⁻⁷ | 0.75 |

TABLE 4B
MOLT-4

| | Ceramides N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | −0.53 | −0.62 | −0.33 | −0.52 | −0.46 | −0.63 | 0.46 | −0.32 | 0.33 | −0.43 |
| Coef. (r) | | | | | | | | | | |
| p | 0.18 | 0.10 | 0.42 | 0.19 | 0.25 | 0.10 | 0.25 | 0.45 | 0.42 | 0.29 |
| Spearman | −0.48 | −0.76 | −0.45 | −0.59 | −0.52 | −0.66 | 0.31 | −0.48 | 0 | −0.55 |
| Coef. (ρ) | | | | | | | | | | |
| p | 0.21 | 0.02 | 0.23 | 0.10 | 0.16 | 0.06 | 0.42 | 0.21 | 0.98 | 0.14 |

TABLE 4C
MOLT-4

| | Total | Total |
|---|---|---|
| | DHCer | Ceramides |
| Pearson | 0.39 | −2.89 × 10⁻³ |
| Coef. (r) | | |
| p | 0.35 | 1.00 |

TABLE 5A
COG-LL-332

| | DHCer N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | 0.06 | 0.45 | −0.16 | 0.32 | −0.09 | −0.54 | 0.73 | 0.07 | 0.74 | 0.14 |
| Coefficient | | | | | | | | | | |
| (r) | | | | | | | | | | |
| p-value | 0.89 | 0.26 | 0.71 | 0.44 | 0.83 | 0.17 | 0.04 | 0.86 | 0.04 | 0.74 |
| Spearman | 0.18 | 0.62 | −0.16 | 0.56 | 0 | −0.42 | 0.81 | 0.62 | 0.83 | 0.45 |
| Coefficient | | | | | | | | | | |
| (ρ) | | | | | | | | | | |
| p-value | 0.62 | 0.09 | 0.66 | 0.12 | 0.98 | 0.26 | 0.01 | 0.09 | 0.01 | 0.23 |

TABLE 5B
COG-LL-332

| | Cer N-acyl chain | | | | | | | | | |
|---|---|---|---|---|---|---|---|---|---|---|
| | C14:0 | C16:0 | C18:0 | C18:1 | C20:0 | C20:1 | C22:0 | C22:1 | C24:0 | C24:1 |
| Pearson | −0.32 | −0.09 | −0.54 | −0.45 | −0.79 | −0.52 | 0.45 | −0.14 | 0.39 | −0.07 |
| Coefficient | | | | | | | | | | |
| (r) | | | | | | | | | | |
| p | 0.44 | 0.82 | 0.17 | 0.26 | 0.02 | 0.19 | 0.27 | 0.75 | 0.34 | 0.86 |
| Spearman | −0.28 | −0.05 | −0.38 | −0.30 | −0.91 | −0.59 | 0 | −0.26 | 0.38 | 0.05 |
| Coefficient | | | | | | | | | | |
| (ρ) | | | | | | | | | | |
| p | 0.46 | 0.89 | 0.32 | 0.42 | 2.0 × 10⁻⁷ | 0.10 | 0.98 | 0.50 | 0.32 | 0.89 |

TABLE 5C
COG-LL-332

| | Total | Total |
|---|---|---|
| | DHCer | Cer |
| Pearson | 0.73 | 0.26 |
| Coef. (r) | | |
| p | 0.04 | 0.53 |
| Spearman | 0.88 | 0.19 |
| Coef. (ρ) | | |
| p | 2.0 × 10⁻⁷ | 0.62 |

### Example 11

### Only Certain Dihydroceramides Correlated with Cytotoxicity

Correlation analysis revealed a significant, strong positive correlation between C22:0- and

C24:0-dihydroceramide levels and cytotoxicity in CCRF-CEM, MOLT-4, COG-LL-317 and COG-LL-332 (Tables 1-5). Cytotoxicity data and absolute sphingolipid levels from treatments to induce both specific dihydroceramide and ceramide accumulation in CCRF-CEM, MOLT-4, COG-LL-317, and COG-LL-332 were analyzed using the Spearman rank correlation method. Shown are coefficients observed to be consistently significant (p<0.05) across the pre-T acute lymphoblastic leukemia cell lines tested. A significant coefficient (ρ) of 0-0.25, 0.25-0.5, and >0.5 represent a weak, moderate, and strong correlation, respectively.

Absolute levels of C22:0- and C24:0-dihydroceramides positively correlated with cytotoxicity in the absence of elevated reactive oxygen species. Due to the confounding effect of in vitro fatty acid conversion, correlation analyses were used to identify significant relationships between absolute levels of specific sphingolipids and cytotoxicity. CCRF-CEM quantitative and cytotoxicity data from treatments that targeted specific sphingolipid synthesis were analyzed with the Spearman non-parametric correlation analysis. A significant, very strong positive correlation between cytotoxicity and absolute levels of both C22:0-DHCer and C24:0-DHCer was observed (see Tables 1-5). Increased reactive oxygen species generation (associated with fenretinide treatment in certain cancer cell lines) was not observed with sphinganine plus GT-11 either with or without C22:0-fatty acid. No consistent correlations were observed between cytotoxicity and total dihydroceramide, total ceramide, or sphingoid base (sphinganine, sphinganine-1-P, sphingosine, sphingosine-1-P) levels. No other dihydroceramides or ceramides levels correlated with cytotoxicity in the T-cell ALL cell lines examined.

### Example 12

### Specific Fatty Acids Increased Fenretinide (4-HPR) Cytotoxicity in Solid Tumor Cell Lines

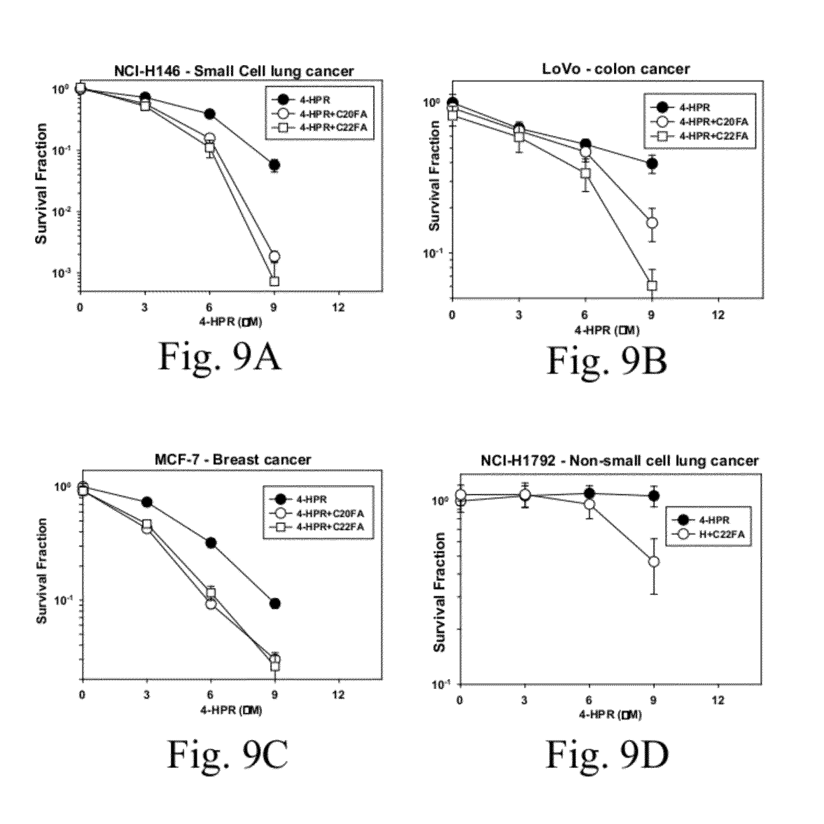

Various solid tumor cell lines of breast and colon cancer, and small cell and non-small cell lung cancers, were exposed to fenretinide with and without exogenous supplementation with nontoxic concentrations (5 micromolar) of single fatty acids (C18:0, C20:0, C22:0 or C24:0) after the manner taken with T-cell ALL cell lines. Results demonstrated (FIGS. 9A-9D) that C20:0 fatty acid increased fenretinide cytotoxicity in LoVo colon cancer and NCI-H146 small cell lung cancer cell lines; C22:0 fatty acid increased fenretinide cytotoxicity in MCF-7 breast cancer cells, NCI-H1792 non-small cell lung and NCI-H146 small cell lung cancer cells, and LoVo colon cancer cells. C18:0 and C24:0 fatty acids did not increase cytotoxicity in any of these cell lines. None of the four fatty acids increased fenretinide cytotoxicity in HT-29 colon cancer cells (not shown). Results indicate that only certain, specific fatty acids can increase fenretinide cytotoxicity in solid tumor cancer cell lines. Taken together with the results observed in four T-cell ALL leukemia cell lines, these results suggest that, unexpectedly, supplementation of fenretinide exposure with certain specific fatty acids can increase cytotoxicity in a cancer cell, or cancer-type, specific manner.

### Example 13

### Only Increase of Certain Dihydroceramides Correlated with Increased Cytotoxicity in Leukemia Cell Lines

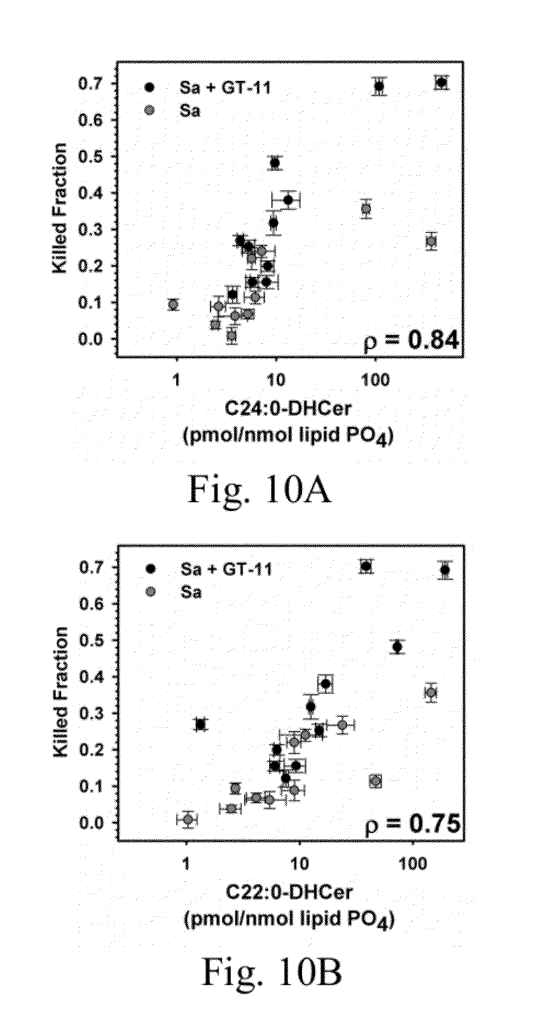

Absolute levels of both C22:0- and C24:0-dihydroceramides positively correlated with cytotoxicity in the CCRF-CEM, MOLT-4, COG-LL-317 and COG-LL-332 T-cell ALL leukemia cell lines. Specifically, a positive, dose-dependent relationship was observed between absolute levels of C22:0- and C24:0-dihydroceramide and cancer cell killed fraction (FIGS. 10A and 10B). No relationship was observed between cytotoxicity and total dihydroceramide, total ceramide, or sphingoid base (sphinganine, sphinganine-1-P, sphingosine, sphingosine-1-P) levels. In addition, C22:0-fatty acid bias of 4-HPR-induced dihydroceramide synthesis resulted in both increased C22:0- and C24:0-dihydroceramide levels (due to metabolism of C22:0 into C24:0 by cellular fatty acid elongases) and markedly increased cytotoxicity.

### DISCUSSION ON EXAMPLES

De novo dihydroceramide synthesis is dependent upon both the expression and regulation of dihydroceramide synthase enzymes as well as fatty acyl-CoA availability. The dihydroceramide synthase enzymes each utilize a specific subset of available fatty acyl-CoAs for de novo dihydroceramide synthesis. This specificity suggests that the sphingolipid fatty acyl chain is physiologically important for function (28). Previous literature has reported dihydroceramides to be non- or minimally cytotoxic to cells, including cancer cells. The present invention discloses evidence that in contrast to previous reports in the scientific literature and, therefore, most unexpectedly, C22:0- and C24:0-dihydroceramides induced dose-dependent cytotoxicity in pre-T acute lymphoblastic leukemia cell lines. Further, results demonstrated that, most unexpectedly, fenretinide-induced cytotoxicity in pre-T acute lymphoblastic leukemia was mediated, in part, through synthesis of C22:0- and C24:0-dihydroceramides and could be increased by exogenous supplementation of these specific fatty acids in the presence of fenretinide. Other examples demonstrate that in other types of cancer cell lines, such as of breast, lung and colon cancers, unexpectedly, fenretinide cytotoxicity could be increased by the exogenous supplementation of specific fatty acids, such as C20:0 and C22:0 fatty acids.

The biochemical model employed to demonstrate the unexpected cytotoxicity of dihydroceramides in the present invention was co-exposure of human cancer cells to minimally-toxic concentrations of exogenous sphinganine, the immediate precursor of dihydroceramides, and GT-11, a specific inhibitor of the conversion of dihydroceramides to ceramides, to drive dihydroceramide synthesis. To manipulate native dihydroceramide levels, solubilized fatty acids were employed to bias the cellular pool of fatty acyl-CoAs utilized by dihydroceramide synthase enzymes. It was hypothesized that supplemented fatty acids would be activated by acyl-CoA synthetase and, through mass effect, bias the pool of cellular fatty acyl-CoAs. Although significant bias of de novo dihydroceramide/ceramide production was observed, the degree of bias was unique for each fatty acyl chain tested.

For example, results demonstrated that the absolute levels of both C22:0- and C24:0-dihydroceramides positively correlated with cytotoxicity in the CCRF-CEM, MOLT-4, COG-LL-317 and COG-LL-332 T-cell ALL leukemia cell lines. Specifically, a dose-dependent relationship was observed between absolute levels of C22:0- and C24:0-dihydroceramide absolute and cell killed fraction. No relationship was observed between cytotoxicity and total dihydroceramide, total ceramide, or sphingoid base (sphinganine, sphinganine-1-P, sphingosine, sphingosine-1-P) or levels of other specific dihydroceramides or ceramides. In addition, C22:0-fatty acid supplementation and resultant production bias of fenretinide (4-HPR)-induced dihydroceramide synthesis resulted in both increased C22:0- and C24:0-dihydroceramide levels and markedly increased cytotoxicity (with C24:0 levels increased by concurrently metabolism of exogenously administered C22:0 fatty acid into C24:0 fatty acid by intracellular fatty acid elongases).

Due to fatty acid modification by cellular elongase and reductase enzymes, increased cytotoxicity with addition of a specific fatty acid to sphinganine or sphinganine/GT-11 could not necessarily be directly interpreted as due to increased levels of the corresponding N-acyl chain ceramide or dihydroceramide. For example, although addition of C20:0-fatty acid resulted in increased sphinganine/GT-11 cytotoxicity in CCRF-CEM, fatty acid elongases metabolized C20:0 currently to C22:0 fatty acid, which also resulted in an increase in C22:0 dihydroceramide, complicating data interpretation. However, statistical correlation analysis of all ALL leukemia cell lines and fatty acid supplementation clearly demonstrated that increased cytotoxicity was due (in the case of C20:0 supplementation) to increased generation of C22:0-dihydroceramide (12-fold increase, P<0.001) (via fatty acid elongation).

The observed significant positive correlations with cytotoxicity for C22:0- and C24:0-dihydroceramides in T-cell ALL cell lines do not exclude that supplementation of other fatty acids, and the resulting increase of the corresponding dihydroceramides, are not preferentially cytotoxic to other cancer cells or other cancer cell types. For example, results taken in various solid tumor cell lines clearly demonstrated indicate that C20:0 fatty acid preferentially increased the cytotoxicity of fenretinide in colon cancer and small cell lung cancer cell lines.

In conclusion, the Examples presented demonstrate that, most unexpectedly, supplementing the exposure of cancer cells to a dihydroceramide-increasing anti-hyperproliferative agent(s), such as fenretinide, with specifically-chosen fatty acids can increase the cytotoxicity of the anti-hyperproliferative agent to the cancer cells to a beneficial effect.

The disclosed methods are generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined herein. Terms defined herein have meanings as commonly understood by a person of ordinary skill in the areas relevant to the present invention. Terms such as “a,” “an,” and “the” are not intended to refer to only a singular entity, but include the general class of which a specific example may be used for illustration. The terminology herein is used to describe specific embodiments of the invention, but their usage does not delimit the disclosed method, except as may be outlined in the claims.

It will be understood that particular embodiments described herein are shown by way of illustration and not as limitations of the invention. The principal features of this invention can be employed in various embodiments without departing from the scope of the invention. The foregoing is illustrative of the present invention, and is not to be construed as limiting thereof.

The invention is defined by the following claims, with equivalents of the claims to be included therein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the methods of use disclosed and claimed herein can be made and executed without undue experimentation in light of the present disclosure. While the methods of this invention have been described in terms of preferred embodiments, it will be apparent to those skilled in the art that variations may be applied to the methods and in the steps or in the sequence of steps of the method described herein without departing from the concept, spirit, and scope of the invention.

More specifically, it will be apparent that certain components which are both related by material and function may be substituted for the components described herein while the same or similar results would be achieved. All such similar substitutes and modifications apparent to those skilled in the art are deemed to be within the spirit, scope, and concept of the invention as defined by the appended claims.

We claim:

1. A method for increasing production of a dihydroceramide having a specific ACYL-chain length and saturation, for improving the effectiveness of a treatment for hyperproliferative cells, the method comprising the steps of:
administering an effective amount of a specific fatty acid; and
administering an effective amount of an anti-hyperproliferative agent;
wherein the specific fatty acid and the anti-hyperproliferative agent increase specific acyl-chained dihydroceramides in the hyperproliferative cells via de novo synthesis in an amount greater than with the anti-hyperproliferative agent alone.

2. The method of claim 1 wherein the anti-hyperproliferative agent is a dihydroceramide-increasing retinoid.

3. The method of claim 2 wherein the retinoid is fenretinide.

4. The method of claim 1 wherein the specific fatty acid administered is chosen from the class of fatty acids with carbon chain length, CX, where X is fourteen to thirty, and saturation, :Y, where Y is zero to six.

5. The method of claim 4 wherein the specific fatty acid is C24:0.

6. The method of claim 4 wherein the specific fatty acid is C22:0.

7. The method of claim 1 wherein the hyperproliferative cells are cancer cells.

8. The method of claim 7 wherein the cancer cells are leukemia, breast cancer, colon cancer, or lung cancer cells.

9. The method of claim 1 wherein the specific fatty acid is administered orally, intravenously, intraarterially, intramuscularly, subcutaneously, intraperitoneally, intravesicularly, intrathecally, sublingually, or topically, in a continuous or discontinuous manner, either before, concurrently with, or after the anti-hyperproliferative agent.

10. The method of claim 1 wherein the specific fatty acid is administered neatly or in a natural product or in a triglyceride or is compounded in a medicant such as a powder, solution, emulsion, liposome, nanoparticle, organized lipid complex, cream, ointment, gel, or salve.

11. The method of claim 1 wherein the specific fatty acid is co-formulated for delivery with the anti-hyperproliferative agent.

12. The method of claim 11 wherein the anti-hyperproliferative agent is fenretinide.

13. The method of claim 1 wherein the specific fatty acid to be administered is determined by a biochemical testing or analysis of the sphingolipid synthetic pathway of the hyperproliferative cells to be treated.

14. The method of claim 1 wherein the specific fatty acid is administered prior to or subsequent to the anti-hyperproliferative agent.
